(19) United States
(12) Patent Application Publication
(10) Pub. No.: US 2006/0134176 A1
(43) Pub. Date: Jun. 22, 2006

(54) Pharmaceutical delivery system and method of use

(75) Inventor: Stephen Bartels, Pittsford, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/314,843

(22) Filed: Dec. 21, 2005

Related U.S. Application Data
(60) Provisional application No. 60/638,875, filed on Dec. 22, 2004.

(51) Int. Cl. A61K 31/407; A61F 2/00

(52) U.S. Cl. 424/427; 514/410

(57) ABSTRACT

The present invention includes a pharmaceutical delivery system comprising a fused pyrrolocarbazole and a pharmaceutical delivery device that is selected from the group consisting of reservoir device and drug infusion device. The pharmaceutical delivery systems are sized and configured to be inserted into the eye of the patient.

### CROSS REFERENCE

This application claims the benefit of Provisional Patent Application No. 60/638,875 filed Dec. 22, 2004 and is incorporated herein by reference.

### BACKGROUND OF THE INVENTION

1. Field of the Invention

The present invention relates generally to pharmaceutical delivery systems, pharmaceutical compositions, methods of use thereof and methods of manufacture thereof for treatment of disease regulated by tyrosine kinase in the ocular region of a patient. More particularly, the present invention relates to pharmaceutical delivery systems, pharmaceutical compositions, methods of use thereof and methods of manufacture thereof for delivering VEGF receptor inhibitors to the ocular region of a patient.

2. Discussion of Related Art

For many years it has been known that treatment of eye disease with a pharmaceutical agent presented challenges because the eye has natural membrane barriers that prevent passage of the pharmaceutical agent into the ocular region. These barriers include the blood-retinal barrier, the cornea, etc. Consequently, systemic treatment of tissue in the eye often requires the level of pharmaceutical agents in the blood plasma to be relatively higher than the therapeutic levels of the pharmaceutical agent in the tissues of the eye to achieve an efficacious result. Application of a pharmaceutical agent topically to the eye also requires passing the pharmaceutical agent through the membrane barriers of the eye such as the cornea. Pharmaceutical agents can be administered to the tissue inside the eye of a patient by a bolus injection. Patients generally dislike the use of bolus injections because of its invasive nature.

Pharmaceutical delivery devices and compositions (i.e. pharmaceutical delivery systems) are currently under development to deliver pharmaceutical agents to the eye of a patient. While placement of a pharmaceutical delivery system is possibly more invasive than a bolus injection, patients expect a pharmaceutical delivery system to deliver the medicament for a longer period of time reducing the requirement for multiple repeated injections into the eye of the patient. Nonetheless, extended release pharmaceutical delivery systems are new, and few medicines can be delivered to the interior portion of the eye by techniques other than a bolus injection.

Examples of extended release pharmaceutical delivery systems are found in US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); U.S. Pat. No. 6,331,313 (Wong et al); and U.S. patent application Ser. No. 10/610,063 (Drug Delivery Device, filed Jun. 30, 2003) (Mosack) all of, which are incorporated by reference. Publications cited throughout this disclosure are incorporated in their entirety herein by reference.

Additionally, US Patent Application Publication 2003/0095995 discloses a formulation for controlled release of drugs by combining hydrophilic and hydrophobic agents. A biodegradable matrix, including polylactate-polyglycolate, is mixed with one or more pharmaceutical agents including corticosteroids and a release modifier. The biodegradable polymer matrix is injected into the eye of a patient and delivers the pharmaceutical agent to the surrounding tissue.

It has been know for some time that tyrosine kinase inhibitors can be used potentially to treat eye disease. U.S. Pat. Nos. 5,980,929, 5,919,813 and WO Publication No. 2000/67,738 discloses the use of genistein as a protein tyrosine kinase pathway inhibitor in the treatment of retinal ischemia, diabetic retinopathy, ocular inflammation, age-related macular degeneration and other ocular disorders. Each of these patents discuss administration by injection in addition to other systemic forms of administration.

Various synthetic small organic molecules that are biologically active and generally known in the art as “fused pyrrolocarbazoles” have been prepared. Examples of such patents include U.S. Pat. Nos. 5,475,110, 5,591,855, 5,594,009, 5,616,724 and 5,705,511. The fused pyrrolocarbazoles were disclosed to be used in a variety of ways, including inhibition of protein kinase C (“PKC”), inhibition of trk tyrosine kinase activity and inhibition of the cellular pathways involved in the inflammation process.

Certain selected fused pyrrolocarbazoles are taught in U.S. Pat. No. 6,630,500 to have activity for inhibition of VEGFR2 as a potential therapeutic for treatment of ocular disease such as retinopathy (including diabetic retinopathy), edema (including macular edema) and ocular inflammation.

U.S. Application Publication No. U.S. 2004/0167091 discloses a biodegradable pharmaceutical delivery system for delivery of anti-VEGF therapy that combines an agent that inhibits the development of neovascularization and particularly an oligonucleotide, with a biodegradable matrix material selected from the group consisting of lactide polymers, lactide/glycolide copolymers, or polyoxyethylene-polyoxypropylene copolymers.

Nonetheless, there is still a need for a drug-delivery system that can be inserted into the eye to deliver a pharmaceutical agent including a tyrosine kinase pathway inhibitor. The present invention addresses these and other needs.

### SUMMARY OF THE INVENTION

The present invention is a pharmaceutical delivery system comprising a fused pyrrolocarbazole and a pharmaceutical delivery device that is sized and configured to be inserted into the eye of the patient. Preferably, the pharmaceutical delivery device is selected from the group consisting of reservoir devices and drug infusion devices.

It has been discovered that the delivery of a fused pyrrolocarbazole with a pharmaceutical delivery device according to the present invention provides sustained prolonged exposure to levels of dosing while avoiding repeated exposure to higher initial concentrations found after a bolus injection. The pharmaceutical delivery system of the present invention controls the amount of fused pyrrolocarbazole in the patient's eye and potentially reduces or eliminates side effects that may result from a bolus injection. In another embodiment, there is a method for treating angiogenic disorders in the eye of a patient, which comprises administering to a host in need of such treatment a pharmaceutical delivery system comprising a pharmaceutical delivery device according to one or more embodiments of the present invention and a therapeutically effective amount of a fused pyrrolocarbazole.

In one embodiment, the fused pyrrolocarbazole is selected from the group consisting of an indolocarbazole and an indenocarbazole and mixtures thereof

In another embodiment, the fused pyrrolocarbazole is a compound defined by the following formula and salts thereof and prodrugs thereof and mixtures of the compound, salt and prodrug thereof: 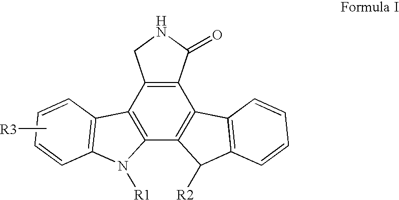
wherein:

R1 and R2 are the same or different and are independently selected from —H, or alkyl of 1-8 carbons, preferably an alkyl of 1-4 carbons, substituted with —OH, or —OR4 where R4 is an alkyl of 1-4 carbons, aryl, preferably phenyl or naphthyl, or the residue of an amino acid after the hydroxyl group of the carboxyl group is removed; and

R3 is —CH₂OH; —CH₂OR7; —(CH₂)ₙSR5; —(CH₂)ₙSO_{y}R5; —CH₂SR₅; or alkyl of 1-8 carbons, preferably an alkyl of 1-4 carbons, substituted with —OH, —OR5, —OR8, —CH₂OR7, —SO_{y}R6 or —SR6; and wherein

R5 is alkyl of 1-4 carbons or aryl, preferably phenyl or naphthyl;

R6 is H, alkyl of 1-4 carbons, aryl of 6-10 carbons, preferably phenyl or naphthyl, or heteroaryl;

R7 is H or alkyl of 1-4 carbons;

R8 is the residue of an amino acid after the hydroxyl group of the carboxyl group is removed;

n is an integer of 1-4; and

y is 1 or 2.

In still another embodiment, the fused pyrrolocarbazole is one or more compounds defined by Formula II and salts thereof and prodrugs thereof and mixtures of the compounds, salts and prodrugs thereof: 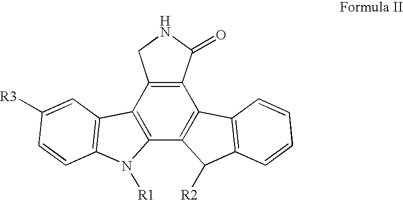
R1 and R₂ are the same or different and are independently selected from H, or alkyl of 1-8 carbons, substituted with —H, —OH or —OR4 where R4 is an alkyl of 1-4 carbons, aryl or the residue of an amino acid after the hydroxyl group of the carboxyl group is removed; and R3 is CH₂OH; —CH₂OR7; —(CH₂)ₙSR5; —(CH₂)ₙSOₘR5; —CH₂SR₅; or alkyl of 1-8 carbons substituted with —OH, —OR5, —OR8, —CH₂OR7, —S(O)ₘR6 or —SR6; and wherein R5 is alkyl of 1-4 carbons or aryl; R6 is H, alkyl of 1-4 carbons or aryl of 6-10 carbons; R7 is H or alkyl of 1-4 carbons; R8 is the residue of an amino acid after the hydroxyl group of the carboxyl group is removed; n is an integer of 1-4; and m is 1 or 2.

In one embodiment, the fused pyrrolocarbazole is defined according to Formula I or Formula II and R1 is an alkyl of 1-4 carbons, substituted with —OH or —OR4 wherein R4 is the residue of an amino acid after the hydroxyl group of the carboxyl group is removed; R2 is H; and R3 is alkyl of 1-4 carbons, substituted with —OR5, —OR8, —CH₂OR7, —S(O)ₘR6 or —SR8; and wherein R5 is alkyl of 1-4 carbons or aryl; R6 is H, alkyl of 1-4 carbons or aryl of 6-10 carbons; R7 is H or alkyl of 1-4 carbons; and R8 is the residue of an amino acid after the hydroxyl group of the carboxyl group is removed.

In another embodiment, there is a fused pyrrolocarbazole as defined in Formula I or Formula II wherein R1 is —CH₂CH₂CH₂OH or —CH₂CH₂CH₂OCOCH₂N(CH₃)₂; R2 is H; and R3 is —CH₂OR7 wherein R7 is alkyl of 1-4 carbons.

In still another embodiment, there is a fused pyrrolocarbazole as defined in mula I or Formula II consisting of compounds represented in Table I (listed below) salts thereof and prodrugs thereof and mixtures of the salts and prodrugs thereof:

TABLE 1

| | |
|---|---|
| | Formula I |
| 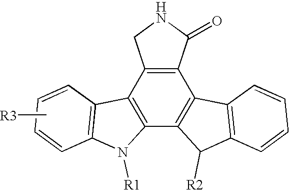 | |

| CMPD | | | |
|---|---|---|---|
| NO | R1 | R2 | R3 |
| 1 | —CH₂CH₂CH₂OH | —H | —CH₂OCH₃ |
| 2 | —CH₂CH₂CH₂OH | —H | —CH₂OCH(CH₃)₂ |
| 3 | —CH₂CH₂CH₂OH | —H | —CH₂O— |
| | | | CH(CH₃)CH₂CH₃ |
| 4 | —CH₂CH₂CH₂OH | —H | (S) —CH₂O— |
| | | | CH(CH₃)CH₂CH₃ |
| 5 | —CH₂CH₂CH₂OH | —H | (R) —CH₂O— |
| | | | CH(CH₃)CH₂CH₃ |
| 6 | —CH₂CHOHCH₃ | —H | —CH₂OCH₂CH₃ |
| 7 | —CH₂CH₂CH₂OH | —H | —CH₂OCH₂CH₂CH₃ |
| 8 | —CH₂CH₂CH₂OH | —H | —CH₂OCH₂CH₂ |
| | | | CH₂CH₃ |
| 9 | —CH₂CH₂CH₂OH | —H | —CH(CH₃)OCH₂CH₃ |
| 10 | —CH₂CH₂CH₂OH | —H | (chiral) |
| | | | —CH(CH₃)OCH₂CH₃ |
| 11 | —CH₂CH₂CH₂OH | —H | (chiral) |
| | | | —CH(CH₃)OCH₂CH₃ |
| 12 | —CH₂CH₂CH₂OH | —H | —CH(CH₃)OCH₃ |
| 13 | —H | | —CH₂OCH₂CH₃ |
| 14 | —CH₂CH₂CH₂OH | —H | —CH(CH₃)O— |
| | | | CH₂CH₂CH₂CH₃ |
| 15 | —CH₂CH₂CH₂OH | —H | —CH(CH₃)O— |
| | | | CH(CH₃)₂ |
| 16 | —CH₂CH₂CH₂OH | —H | —CH₂OC(CH₃)₃ |
| 17 | —CH₂CH₂CH₂OCO— | —H | —CH₂OCH(CH₃)₂ |
| | CH₂NH₂ | | |
| 18 | —CH₂CH₂CH₂OCO— | —H | —CH₂OCH(CH₃)₂ |
| | CH₂NH₂— | | |
| | CH₂CH₂CH₂CH₂NH₂ | | |
| 19 | —CH₂CH₂CH₂OCOCH₂— | —H | —CH₂OCH(CH₃)₂ |
| | CH₂NH₂ | | |
| 20 | —CH₂CH₂CH₂OCOCH₂— | —H | —CH₂OCH(CH₃)₂ |
| | CH₂CH₂N(CH₃)₂ | | |
| 21 | —CH₂CH₂CH₂OCO— | —H | —CH₂OCH(CH₃)₂ |
| | CH₂N(CH₂)₂ | | |
| 22 | —CH₂CH₂CH₂OCO— | —H | —CH₂OCH(CH₃)₂ |
| | CH₂CH₂CH₂ | | |
| 23 | —CH₂CH₂OH | —H | —CH₂SCH₂CH₃ |
| 24 | —CH₂CH₂CH₂OH | —H | —CH₂SCH₂CH₃ |
| 25 | —CH₂CH₂CH₂OH | —H | —CH₂S(O)CH(CH₃)₂ |
| 26 | —CH₂CH₂OH | —H | —CH₂OH |
| 27 | —H | —H | —CH₂OH |
| 28 | —H | —H | —CH₂OCH₂CH₃ |
| 29 | —H | —H | —CH₂OCH(CH₃)₂ |
| 30 | —CH₂CH₂CH₂OH | —H | —CH(OH)CH₃ |
| 31 | —CH₂CH₂CH₂OH | —H | —CH(OH)CH₂CH₃ |
| 32 | —H | —H | —CH(OH)CH₃ |
| 33 | —H | —H | (+/−) |
| | | | —CH(OCH₃)CH₃ |
| 34 | —CH₂CH₂CH₂OH | —CH₂OH | —CH₂OCH(CH₃)₂ |

In another embodiment, the fused pyrrolocarbazole is of the following formula and salts thereof and prodrugs thereof and mixtures of the compound, salts and prodrugs thereof:

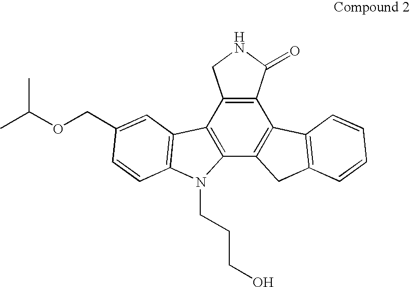

In still another embodiment, the fused pyrrolocarbazole is a compound of the following formula and salts thereof and prodrugs thereof and mixtures of the compound, salts and/or prodrugs thereof:

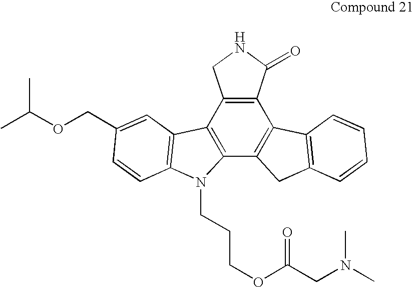

### BRIEF DESCRIPTION OF THE DRAWING

FIG. 1 of the present invention is an enlarged cross-sectional view down the center of one embodiment of a sustained release pharmaceutical delivery system.

FIG. 2 is a cross sectional view of a first embodiment of a pharmaceutical delivery system of this invention.

FIG. 3 is a second cross-sectional view of the device of FIG. 1 viewed along the line 3-3.

### DETAILED DESCRIPTION OF PREFERRED EMBODIMENTS

The present invention is a pharmaceutical delivery system comprising a fused pyrrolocarbazole and a pharmaceutical delivery device that is sized and configured to be inserted into the eye of the patient. Preferably, the pharmaceutical delivery device is selected from the group comprising reservoir devices and drug infusion devices. It has been discovered that the delivery of a fused pyrrolocarbazole with a pharmaceutical delivery system provides sustained prolonged exposure to levels of dosing while avoiding repeated exposure to higher initial concentrations found after a bolus injection. The pharmaceutical delivery system controls the amount of fused pyrrolocarbazole in the patient's eye and potentially reduces or eliminates side effects that may result from a bolus injection. In another embodiment, there is a method for treating angiogenic disorders in the eye of a patient, which comprises administering to a host in need of such treatment a pharmaceutical delivery system comprising a pharmaceutical delivery device and a therapeutically effective amount of a fused pyrrolocarbazole.

Definitions

“Pharmaceutically acceptable salts” is defined as a salt formed by addition of an acid to a base containing organic molecule or a base to an acid containing organic molecule.

“Fused pyrrolocarbazole” is defined as a compound having a fused pyrrolocarbazole core structure as shown in the following Formula IV: 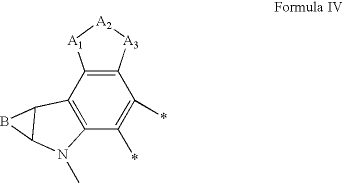
wherein at least one of A1, A2 or A3 is a nitrogen B is a structure that forms an aryl or heteroaryl ring systems with the carbon atoms to, which B is bonded. The designation * indicates the attachment point of an additional fused ring system.

The core structures provided herein are presented by way of the general guidance and are not to be taken as limiting the scope of the invention. For example, certain cores indicate the presence of certain atoms for illustrative purposes. It will be appreciated that such atoms may be bonded to additional groups, or may be further substituted without deviating from the spirit of the invention.

Thus, fused pyrrolocarbazole core structures include, but are not limited to, structures of formula V as follows: 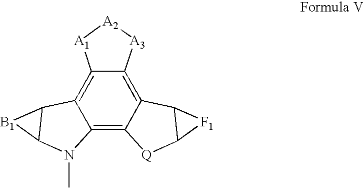
wherein at least one of A1, A2 and A3 is a nitrogen, B1 and F1 together with the adjacent carbons to, which they are attached independently form an aryl or heteroaryl ring. Q is a moiety containing one or more nitrogen atoms or carbon atoms. Such structures include but are not limited to indolocarbazoles, indenocarbazoles and bridged indenocarbazoles.

As used herein, “indolocarbazole” is intended to indicate a compound of formula V, wherein at least one of A1, A2 and A3 is a nitrogen. B1 and F1 together with the adjacent carbons to, which they are attached independently form an aryl or heteroaryl ring. Q is nitrogen.

As used herein, “indenocarbazole” is intended to indicate a compound of formula V, wherein at least one of A1, A2 and A3 is a nitrogen. B1 and F1 together with the adjacent carbons to, which they are attached independently form an aryl or heteroaryl ring. Q is a substituted or unsubstituted carbon atom.

“Inflammation-mediated condition of the eye” is defined as any condition of the eye, which may benefit from treatment with an anti-inflammatory agent and is meant to include, but is not limited to, uveitis, macular edema, acute macular degeneration, retinal detachment, ocular tumors, fungal or viral infections, multifocal choroiditis, diabetic uveitis, proliferative vitreoretinopathy (PVR), sympathetic opthalmia, Vogt Koyanagi-Harada (VKH) syndrome, histoplasmosis and uveal effusion.

“Angiogenesis-mediated condition of the eye” is defined as any condition of the eye that is caused by the pathway for growth of new blood vessels. Some angiogenesis-mediated condition of the eye includes but are not limited to ocular neovascularization including neovascularization of the cornea, iris, retina, as well as choroidal neovascularization associated with histoplasmosis, pathological myopia, age-related macular degeneration, angioid streaks, anterior ischemic optic neuropathy, bacterial endocarditis, Best's disease, birdshot retinochoroidopathy, choroidal hemangioma, choroidal nevi, choroidal nonprofusion, choroidal osteomas, choroidal rupture, choroderemia, chronic retinal detachment, coloboma of the retina, drusen, endogenous Candida endophthalmitis, extrapapilary hamartoma of the retinal pigmented epithelium, fundus flavimaculatus, idiopathic macular hole, malignant melanoma, metallic intraocular foreign body, morning glory disc syndrome, multiple evanescent, white-dot syndrome, neovascularization at ora serrata, operating microscope burn, optic nerve head pits, photocoagulation, punctuate inner choroidopathy, radiation retinopathy, retinal cryoinjury, retinitis pigmentosa, retinochoroidal coloboma, rubella, sarcoidosis, serpiginous or geographic choroiditis, subretinal fluid drainage, tilted disc syndrome, Taxoplasma retinchoroiditis, tuberculosis or Vogt-Koyanagi-Harada syndrome.

The terms, “inhibit” and “inhibition” are defined as a specified response of a designated material (e.g., enzymatic activity) is comparatively decreased in the presence of a fused pyrrolocarbazole of the present invention.

The term “contacting” is defined as directly or indirectly causing placement together of two items, such that the two items directly or indirectly come into a physical or chemical association with each other to affect a particular outcome.

As used herein, “prodrug” is intended to include any covalently bonded carrier, which releases the active parent pharmaceutical agent as a compound of the present invention in vivo when such prodrug is administered to a mammalian subject. Since prodrugs are known to enhance numerous desirable qualities of pharmaceuticals (e.g., solubility, bioavailability, manufacturing, etc.) the compounds of the present invention may be delivered in prodrug form. Thus, the present invention contemplates prodrugs of the compounds of the present invention, compositions containing the same and methods of treating diseases and disorders with such prodrugs. Prodrugs of a compound of the present invention, for example Formula I, may be prepared by modifying functional groups present in the compound in such a way that the modifications are cleaved, either in routine manipulation or in vivo, to the parent compound. Accordingly, prodrugs include, for example, compounds of the present invention wherein a hydroxy, amino, or carboxy group is bonded to any group that, when the prodrug is administered to a mammalian subject, cleaves to form a free hydroxyl, free amino, or carboxylic acid, respectively. Examples include, but are not limited to, the residue of an amino acid after the hydroxyl group of the carboxyl group is removed acetate, formate and benzoate derivatives of alcohol and amine functional groups; and alkyl, carbocyclic, aryl and alkylaryl esters such as methyl, ethyl, propyl, iso-propyl, butyl, isobutyl, sec-butyl, tert-butyl, cyclopropyl, phenyl, benzyl and phenethyl esters and the like.

Certain abbreviations used to delineate the results below are defined as follows: “μg” denotes microgram, “mg” denotes milligram, “g” denotes gram, “μL” denotes microliter, “mL” denotes milliliter, “L” denotes liter, “nM” denotes nanomolar, “μM”0 denotes micromolar, “mM” denotes millimolar, “M” denotes molar and “nm” denotes nanometer.

A minimum of about ½ of the particles has a particle size less than 200 microns. “Reservoir device” is defined as a pharmaceutical delivery device that has a core that receives a pharmaceutical agent that is at least in part encapsulated by a barrier that limits the rate of release of the pharmaceutical agent from the reservoir delivery system and extends the duration of release of the pharmaceutical agent. “Drug Infusion Device” is a device that releases a pharmaceutical agent from a non-drug permeable reservoir through one or more ports by means of a pressure differential inside the reservoir and outside the reservoir. Drug infusion systems include osmotic pumps.

Active Ingredients

One embodiment of the present invention is the fused pyrrolocarbazoles represented by Formula I:

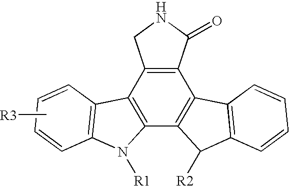

wherein:

R1 and R2 are the same or different and are independently selected from H, or alkyl of 1-8 carbons, preferably an alkyl of 1-4 carbons, substituted with —OH, or —OR4 where R4 is an alkyl of 1-4 carbons, aryl, preferably phenyl or naphthyl, or the residue of an amino acid after the hydroxyl group of the carboxyl group is removed; and

R3 is —CH₂OH; —CH₂OR7; —(CH₂)ₙSR5; —(CH₂)ₙSO_{y}R5; —CH₂SR₅; or alkyl of 1-8 carbons, preferably an alkyl of 1-4 carbons, substituted with —OH, —OR5, —OR8, —CH₂OR7, —SO_{y}R6 or —SR6. R5 is alkyl of 1-4 carbons or aryl, preferably phenyl or naphthyl. R6 is H, alkyl of 1-4 carbons, aryl of 6-10 carbons, preferably phenyl or naphthyl, or heteroaryl. R7 is H or alkyl of 1-4 carbons. R8 is the residue of an amino acid after the hydroxyl group of the carboxyl group is removed; n is an integer of 1-4; and y is 1 or 2.

In certain preferred embodiments, the compounds of Formula I are those of Formula II:

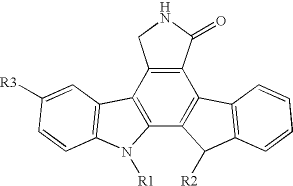

wherein R1, R2 and R3 are as defined for Formula I above.

In certain referred embodiments, R1 is an alkyl of 1-4 carbons, substituted with —OH or —OR4 where R4 is an alkyl of 1-4 carbons (inclusive), aryl, preferably phenyl or naphthyl, or the residue of an amino acid after the hydroxyl group of the carboxyl group is removed. R₂ is H; and R₃ is —CH₂OH; —CH₂OR7; —(CH₂)ₙSR5; —(CH₂)ₙS(O)_{y}R5; —CH₂SR₅; or alkyl of 1-8 carbons, preferably an alkyl of 1-4 carbons, substituted with —OH, —OR5, —OR8, —CH₂OR7, —S(O)_{y}R6 or —SR6. R5, R6, R7 and R8 are as defined for Formula I above.

In certain other preferred embodiments, R1 is —CH₂CH₂CH₂OH or —CH₂CH₂CH₂—OCOCH₂N(CH₃)₂, R2 is H and R3 is —CH₂OR₇; wherein R7 is alkyl of 1-4 carbons.

In certain even further preferred embodiments the fused pyrrolocarbazoles of Formula I and/or Formula II are those represented in Table I.

Particularly preferred compounds of Table 1 include compounds 1, 2, 3, 4, 5, 6 and 21 with compounds 2 and 21 being most preferred.

Pharmaceutically acceptable salts of the fused pyrrolocarbazoles of the present invention also fall within the scope of the compounds as disclosed herein. Some examples of acid addition salts include the hydrochloride, sulfate and phosphate salts of a base containing organic molecule. Some examples of organic acid addition salt such as acetate, maleate, fumarate, tartrate and citrate salts of the base containing organic molecule. Examples of pharmaceutically acceptable metal salts are alkali metal salts such as sodium salt and potassium salt, alkaline earth metal salts such as magnesium salt and calcium salt, aluminum salt and zinc salt. Examples of pharmaceutically acceptable ammonium salts are ammonium salt and tetramethylammonium salt. Examples of pharmaceutically acceptable organic amine addition salts are salts with morpholine and piperidine. Examples of pharmaceutically acceptable amino acid addition salts are salts with lysine, glycine and phenylalanine.

Therapeutic and Prophylactic Indications

In one embodiment, there is a pharmaceutical delivery system for treating inflammation-mediated condition of the eye, for example edema. The pharmaceutical delivery system comprises, a fused pyrrolocarbazole, an indenocarbazole, an indolocarbazole, a compound of Formula I, a compound of Formula II or a compound of Table I and a pharmaceutical delivery device according to any one of the embodiments disclosed herein.

In one embodiment, there is a pharmaceutical delivery system for inhibiting VEGFR kinase activity in the eye. The pharmaceutical delivery system comprises, a fused pyrrolocarbazole, an indenocarbazole, an indolocarbazole, a compound of Formula I, a compound of Formula II or a compound of Table I and a pharmaceutical delivery device according to any one of the embodiments disclosed herein.

In one embodiment, there is a pharmaceutical delivery system for treating angiogenesis disorders in the eye of a patient. The pharmaceutical delivery system comprises, a fused pyrrolocarbazole, an indenocarbazole, an indolocarbazole, a compound of Formula I, a compound of Formula II or a compound of Table I and a pharmaceutical delivery device according to any one of the embodiments disclosed herein.

In one embodiment, there is a method of treating inflammation-mediated condition (for example edema) of the eye. The method comprising administering to the eye of a patient a pharmaceutical delivery system comprises, a fused pyrrolocarbazole, an indenocarbazole, an indolocarbazole, a compound of Formula I, a compound of Formula II or a compound of Table I and a pharmaceutical delivery device according to any one of the embodiments disclosed herein.

In one embodiment, there is a method for inhibiting VEGFR kinase activity in the eye of a patient. The method comprises administering to the eye of a patient a pharmaceutical delivery system comprising, a fused pyrrolocarbazole, an indenocarbazole, an indolocarbazole, a compound of Formula I, a compound of Formula II or a compound of Table I and a pharmaceutical delivery device according to any one of the embodiments disclosed herein.

In one embodiment, there is a pharmaceutical delivery system for treating angiogenesis disorders in the eye of a patient. The method comprises administering to a patient a pharmaceutical delivery system comprises, a fused pyrrolocarbazole, an indenocarbazole, an indolocarbazole, a compound of Formula I, a compound of Formula II or a compound of Table I and a pharmaceutical delivery device according to any one of the embodiments disclosed herein.

In one embodiment, there is a pharmaceutical delivery system for treating retinopathy, diabetic retinopathy or macular degeneration in the eye of a patient. The pharmaceutical delivery system comprises, a fused pyrrolocarbazole, an indenocarbazole, an indolocarbazole, a compound of Formula I, a compound of Formula II or a compound of Table I and a pharmaceutical delivery device according to any one of the embodiments disclosed herein.

In one embodiment, there is a pharmaceutical delivery system for treating retinopathy, diabetic retinopathy or macular degeneration in the eye of a patient. The pharmaceutical delivery system comprises, a fused pyrrolocarbazole, an indenocarbazole, an indolocarbazole, a compound of Formula I, a compound of Formula II or a compound of Table I having a single or multiple crystalline morphology and a pharmaceutical delivery device according to any one of the embodiments disclosed herein.

The fused pyrrolocarbazoles of the present invention have important functional pharmacological activities, which find utility in a variety of settings, including both research and therapeutic arenas. For ease of presentation and in order not to limit the range of utilities for, which these compounds can be characterized, we generally describe the activities of the fused pyrrolocarbazoles in ocular tissue including inhibition of enzymatic activity such as the enzymatic kinase activity of VEGFR1 and VEGFR2; and inhibition of inflammation-associated responses.

Synthesis

The present invention also provides a method for preparing the fused pyrrolocarbazoles of the present invention. The compounds of the present invention may be prepared in a number of ways well known to those skilled in the art. Specifically, Compounds A and B were prepared according to the disclosure of U.S. Pat. Nos. 5,475,110, 5,591,855, 5,594,009, 5,616,724, 5,705,511 and 6,630,500, which is incorporated herein by reference in its entirety.

It will be appreciated that the compounds of the present invention may contain one or more asymmetrically substituted carbon atoms and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare such optically active forms. For example, mixtures of stereoisomers may be separated by standard techniques including, but not limited to, resolution of racemic forms, normal, reverse-phase and chiral chromatography, preferential salt formation, recrystallization and the like, or by chiral synthesis either from active starting materials or by deliberate chiral synthesis of target centers.

As will be readily understood, functional groups present on the compounds of the present invention may contain protecting groups. For example, the amino acid side chain substituents of the compounds can be substituted with protecting groups such as benzyloxycarbonyl or tert-butoxycarbonyl groups. Protecting groups are known per se as chemical functional groups that can be selectively appended to and removed from functionalities, such as hydroxyl groups and carboxyl groups. These groups are present in a chemical compound to render such functionality inert to chemical reaction conditions to, which the compound is exposed. Any of a variety of protecting groups may be employed with the present invention. Preferred protecting groups include the benzyloxycarbonyl (Cbz; Z) group and the tert-butyloxycarbonyl (Boc) group. Other preferred protecting groups according to the invention may be found in Greene, T. W. and Wuts, P. G. M., “Protective Groups in Organic Synthesis” 2d. Ed., Wiley & Sons, 1991.

Combination Therapies

The fused pyrrolocarbazole may be administered in combination with one or more additional pharmaceutical agents, such as the individual compounds and therapeutic agent within one or more of the therapeutic classes selected from the group comprising anti-metabolites, anti-biotics, antibacterials, antifungal antibiotics, synthetic antifungals, steroids, anti-proliferative agents, matrix metalloproteinase inhibitors, thrombolytic agents, anti-neoplastic agents, non-steriodal anti-inflammatories (NSAIDS) and retinoids.

Additionally, the fused pyrrolocarbazoles of the present invention optionally can be used in combination with one or more anti-angiogenesis agents including but not limited to other tyrosine kinase inhibitors, inhibitors of growth factors, inhibitors of Tie-2, inhibitors of angiopoetin.

Additionally, the fused pyrrolocarbazoles of the present invention can be used in combination with agents that promote survival of retinal cells including, but not limited to, neurons, glia and retinal pigment epithelium, such as neurotrophic factors, anti-apoptosis agents, and anti-caspase agents.

The fused pyrrolocarbazole or the fused pyrrolocarbazole and the additional pharmaceutical agent(s) are preferably from about 10 to 90% by weight of the pharmaceutical delivery system. More preferably, the fused pyrrolocarbazole or the fused pyrrolocarbazole and the additional pharmaceutical agent(s) are from about 50 to about 80% by weight of the pharmaceutical delivery system. In a preferred embodiment, the agent comprises about 50% by weight of the pharmaceutical delivery system. In a particularly preferred embodiment, the agent comprises about 70% by weight of the pharmaceutical delivery system.

Reservoir Delivery Device

In another embodiment, the delivery device is a reservoir delivery device.

In another embodiment, the reservoir delivery device has a drug impermeable portion that surrounds at least a portion of a drug core comprising a fused pyrrolocarbazole, wherein the drug impermeable portion defines a reservoir and restricts flow of fused pyrrolocarbazole from the reservoir.

In still another embodiment, the drug impermeable portion defines a wall, layer or coating. Typically, the drug impermeable portion is made of silicone. In another embodiment, the reservoir device is made at least in part of an impermeable polymer material. The permeable polymer material covers, houses, coats or encapsulates at least a portion of the drug core.

In another embodiment the reservoir delivery device having a drug impermeable portion a drug permeable portion that surrounds at least a portion of a drug core. The drug impermeable portion further defines the reservoir and allows passage of fused pyrrolocarbazole through the drug permeable portion. Typically, the drug permeable portion defines a wall, layer or coating.

Optionally, the drug permeable portion is made of poly(vinyl alcohol).

In still another embodiment, there is a pharmaceutical delivery system that comprises a holder made of a material impermeable to passage of a pharmaceutical agent and including at least one opening for passage of the active agent therethrough. The drug core is contained in the holder. The drug core includes fused pyrrolocarbazole. The pharmaceutical delivery system optionally includes a preformed disc made of a material that is permeable to passage of the fused pyrrolocarbazole. The preformed disc is located, preferably, between the drug core and the opening.

In one embodiment, there is no preformed disc that is permeable to the passage of the fused pyrrolocarbazole. In this embodiment, it is understood, typically, that the release rate of the fused pyrrolocarbazole is affected by the size of the opening. In the embodiment with a preformed fabricated disc, the disc is contained in the holder and disposed between the drug core and the at least one opening in the holder. Optionally, a groove is formed in the holder in the vicinity of the disc. When the preformed fabricated disc is placed between the drug core and the opening, it is understood that the permeability of the preformed disc effects the rate of release of the fused pyrrolocarbazole

The size and thickness of the permeable membrane determines the rate of diffusion and delivery of the pharmaceutical agent in a reservoir delivery device. The permeable membrane can be a coating applied directly to the surface of all or a portion of the surface of the therapeutically active agent or all or part of a preformed hosing that surrounds the therapeutically active agent. Examples of devices with permeable membranes are found in US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Pharmaceutical Delivery Device, filed Mar. 28, 2003) (Mosack et al.); and U.S. patent. application Ser. No. 10/610,063 (Pharmaceutical Delivery Device, filed Jun. 30, 2003) (Mosack) all of, which are incorporated by reference.

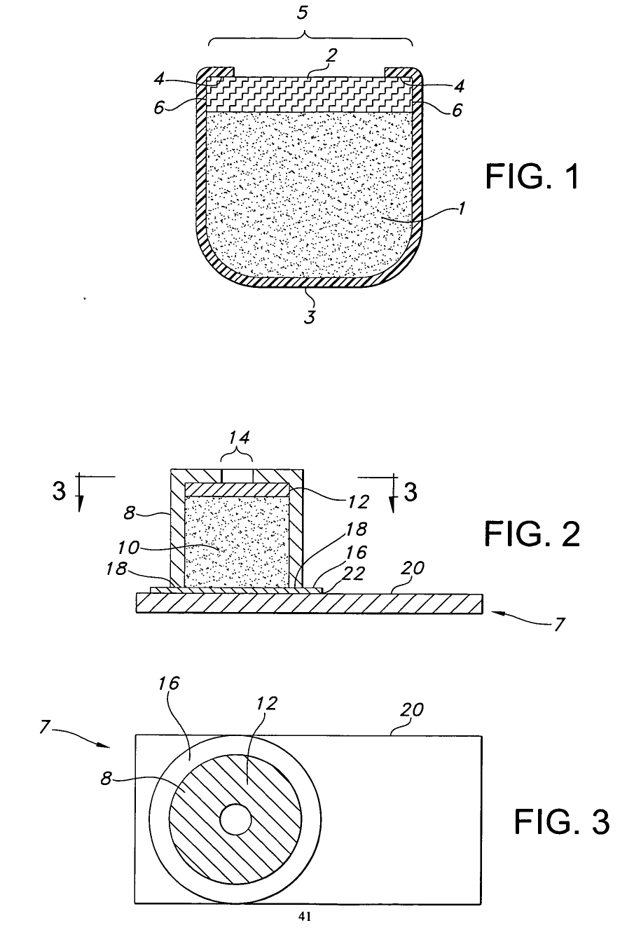

Without limiting the invention to a particular embodiment, FIG. 1 illustrates one type of reservoir device. The reservoir is defined by a cup 3 that is made of an impermeable material and contains a drug core 1. The drug core is made at least in part made of a therapeutically active agent. The cup 3 has one or more lips 4 extending inward around the open top end 5 of the cup 3. Optionally, a prefabricated plug 2 formed of a material that is permeable to the fused pyrrolocarbazole (including poly (vinyl alcohol)) is positioned in the recess between the top end of the drug core 1 and below the one or more lips 4 such that the one or more lips 4 interact with the prefabricated plug 2 holding it in position and closing the open top end 5 of the cup 3. It is understood by a person of ordinary skill in the art that when the prefabricated plug 2 is placed in the recess between the drug core 1 and below one or more lips 4 at the top end 5 of the cup 3, the permeability of the prefabricated plug 2 will effect the rate of release of the fused pyrrolocarbazole. In an embodiment where there is no prefabricated plug 2, the size of the opening adjacent the open top end 5 determines the rate of release of the fused pyrrolocarbazole.

The one or more lips 4 are made of the same impermeable material as the unitary cup 3 and protrude inwardly from the top open end 5 of the cup 3. In one embodiment, the cup 3 and lips 4 are formed in a single unitary design to provide structural integrity to the device and facilitate manufacturing and handling. The lips 4 are designed to enable the prefabricated plug 2 to snap into place and then to hold the plug 2 in place during use. They can vary in size or shape. The lips 4 of the present invention include nubs, tabs, ridges and any other raised or protruding member.

By prefabricating the permeable plug 2 it can be snapped into or securely placed in the device in one step. The prefabricated plug 2 can be fabricated or machined to various dimensional specifications, which can be used to control diffusion properties to achieve a desired release rate. The same unitary cup and lips design can be used for pharmaceutical delivery systems with a variety of release rates making it possible to use a single manufacturing line or type of equipment. Thus, the present invention allows for ease of construction by more standard manufacturing techniques into systems with different release rates.

Together the cup 3 with lips 4 and the prefabricated permeable plug 2 acts as a reservoir surrounding the drug core 1 to keep the drug core in place. The therapeutically active agent diffuses out of the drug core 1, through the prefabricated permeable plug 2 and out of the open top end 5. The prefabricated plug 2 has substantially the same radial extent as the cup 3, so that the only diffusion pathway is out of the plug 2 and not around the sides 6. Glue, a polymeric substance or other adhesion means can be employed to further bond the plug to the cup.

For one embodiment, the fused pyrrolocarbazole is optionally provided in the form of a micronized powder and then mixed with an aqueous solution of bioerodible polymer, whereby the fused pyrrolocarbazole and bioerodible polymer agglomerate into larger sized particles. The resulting mixture is then dried to remove some of the moisture and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus, this tablet representing inner drug core 1.

Another device is made according to one embodiment of the present invention is described with reference to FIGS. 2 and 3. Device 7 is a sustained release pharmaceutical delivery system for implanting in the eye. Device 7 includes inner drug core 10 including a therapeutically active agent.

As shown in FIGS. 2 and 3, therapeutically active agent, optionally, is mixed with a matrix material to effectively bind the therapeutically active agent into a tablet form for easy insertion into the pharmaceutical delivery system 7. Preferably, the matrix material is a polymeric material that is compatible with body fluids and the eye. Additionally, matrix material should be permeable to passage of the therapeutically active agent therethrough, particularly when the device is exposed to body fluids. In one embodiment, the matrix material is PVA. Also, in one embodiment, the therapeutically active agent is optionally coated with a coating permeable polymeric material, which is the same or different from material mixed with the therapeutically active agent.

The pharmaceutical delivery system 7 includes a cup shape holder 8 for the inner drug core 10. Holder 8 is made of a material that is impermeable to passage of the therapeutically active agent. Since holder 8 is made of the impermeable material, an opening 14 is formed in holder 8 to permit the therapeutically active agent to pass through the opening 14 and contact the surrounding eye tissue. Optionally a drug permeable membrane 12 is positioned in the holder 8 between the drug core 10 and the opening 14 to further effect the rate of release of the therapeutically active agent from the pharmaceutical delivery system 7. In an embodiment without a drug permeable membrane, the rate of release is affected by the size of the opening 14. In an embodiment with a drug permeable membrane, the rate of release is affected by the permeability of the drug permeable membrane.

The cup shaped holder 8, of one embodiment, has a mouth portion opposite the opening 14 that is configured, optionally, to receive the drug permeable membrane 12 and the drug core 10 during manufacture. A sealable lid 16 is placed over the mouth of the holder and sealed by an adhesive layer 18. The sealable lid 16, of one embodiment, is made of an impermeable material such as silicone. The sealable lid 16 optionally has an extended portion such as a suture tab (not shown in this form) that is configured to suture the pharmaceutical delivery system to adjacent tissue in the patient's eye according to techniques that are recognized in the art.

In the illustrated embodiment, a suture tab 20 is affixed to the lid 16 by an adhesive layer 22. The suture tab 20, illustrated in the present invention, is made of poly(vinyl alcohol).

In one embodiment, the preformed disk is made of poly(vinyl alcohol). In assembling this embodiment, a solution of uncured poly(vinyl alcohol) is distributed evenly and dried into uncured poly(vinyl alcohol) sheets. The sheets are placed into an oven or drier for curing.

The pharmaceutical delivery system of FIGS. 2 and 3 is made by providing an impermeable cup shaped holder 8. The cup shape holder 8 has an opening 14 that is sized and configured to effect the rate of release of the therapeutically active agent from the pharmaceutical delivery system. The holder 8 also has a mouth. Typically, the mouth is larger than the opening 14. A drug permeable membrane 12 is inserted through the mouth into the cup-shaped holder 8 and is positioned in a covering relationship with the opening 14. Typically, the membrane sealably covers the opening 14. Optionally, the membrane is adhered to the holder 8.

Thereafter, the drug core 10 is inserted into the cup shaped holder 8 adjacent the drug permeable membrane. Thereafter, a sealable lid 16 is adhered to the cup shaped holder 8 by an adhesive layer 18. The sealable lid 16 is made of a drug impermeable material such as silicone. Optionally, a suture tab 20 is affixed to the sealable lid 16 by an adhesive layer 22. Suture tab 20 is drug permeable or drug impermeable. In the present invention, the suture tab 20 is made of poly(vinyl alcohol).

The formulation of the pharmaceutical delivery systems for use in the invention may vary according to the preferred drug release profile, the particular therapeutically active agent, the condition being treated and the medical history of the patient.

In one embodiment, the pharmaceutical delivery system is configured to maintain the concentration of fused pyrrolocarbazole in the vitreous that is a minimum of about 10 ng/ml. In still another embodiment, there is a method of treating a patient that includes maintaining the concentration of fused pyrrolocarbazole in the vitreous that is a minimum of about 10 ng/ml. Typically, the concentration of fused pyrrolocarbazole in the vitreous is a minimum of about 50 ng/ml, 100 ng/ml, 500 ng/ml or 1000 ng/ml and or a maximum of about 10 mg/ml, 5 mg/ml, 1 mg/ml or 500 μg/ml.

In another embodiment, the pharmaceutical delivery system is configured to maintain the effective concentration of fused pyrrolocarbazole in the vitreous that is at least about 50 times greater than the concentration of the fused pyrrolocarbazole in the blood of the patient. Typically, the effective concentration is at least about 100, about 200, about 500 or about 1000 times greater than the concentration of the same VEGF inhibitor in the blood of the patient.

In one embodiment, there is an effective concentration of fused pyrrolocarbazole in the vitreous that is maintained for a minimum of 6 weeks. Generally, the effective concentration of VEGF inhibitor is maintained in the vitreous of the patient for a period of a minimum of 8 weeks, 12 weeks, 6 months, I year, 2 years or 3 years.

In another embodiment, the fused pyrrolocarbazole is released from the pharmaceutical delivery system at rate that is a minimum of about 5 ng per day and a maximum of about I mg per day. Typically, the anti-angiogenesis agent is released from the pharmaceutical delivery system at rate that is a minimum of about 10 ng per day, about 50 ng per day, about 100 ng per day or about 500 ng per day and/or a maximum of 1 μg, 100 μg per day, 500 μg per day or 1 mg per day.

Kits for the Administration of the Biodegradable Polymer Matrices

In another aspect of the invention, kits for treating angiogenesis-mediated conditions of the eye, comprising: (a) one or more pharmaceutical delivery systems disclosed herein and (b) instructions for use.

In another aspect of the invention, kits for treating one or more inflammatory conditions of the eye (for example edema) that are identified herein that are provided, comprising: (a) one or more pharmaceutical delivery systems disclosed herein and (b) instructions for use.

Method of Administering Pharmaceutical Delivery Systems

The pharmaceutical delivery systems are typically inserted into the eye by a trocar following making an incision in the sclera sized to receive the trocar. The method of placement may influence the pharmaceutical agent release kinetics. For example, implanting the device with a trocar may result in placement of the device deeper within the vitreous than placement by forceps, which may result in the biodegradable polymer matrix being closer to the edge of the vitreous. The location of the implanted device may influence the concentration gradients of pharmaceutical agent surrounding the device and thus influence the release rates (e.g., a device placed closer to the edge of the vitreous will result in a slower release rate). One example of a placement device is found in U.S. Patent Publ. No. 2003/0135153, which is incorporated herein by reference in its entirety.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

What is claimed is:

1. A pharmaceutical delivery system comprising a fused pyrrolocarbazole and a pharmaceutical delivery device selected from the group consisting of reservoir devices and drug infusion device, wherein the pharmaceutical delivery device is sized and configured to be inserted into the eye of the patient.

2. The pharmaceutical delivery system of claim 1, wherein the fused pyrrolocarbazole is selected from the group consisting of an indolocarbazole and an indenocarbazole and mixtures thereof.

3. The pharmaceutical delivery system of claim 1, wherein the fused pyrrolocarbazole is a compound defined by the following formula and salts thereof and prodrugs thereof and mixtures of the compound, salt and prodrug thereof: 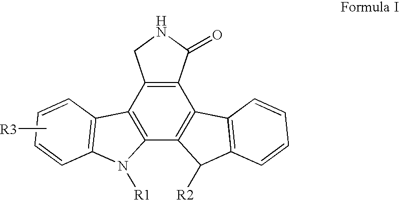
wherein:
R1 and R2 are the same or different and are independently selected from —H, or alkyl of 1-8 carbons, preferably an alkyl of 1-4 carbons, substituted with —OH, or —OR4 where R4 is an alkyl of 1-4 carbons, aryl, preferably phenyl or naphthyl, or the residue of an amino acid after the hydroxyl group of the carboxyl group is removed; and
R3 is —CH₂OH; —CH₂OR7; —(CH₂)ₙSR5; —(CH₂)ₙSO_{y}R5; —CH₂SR₅; or alkyl of 1-8 carbons, preferably an alkyl of 1-4 carbons, substituted with —OH, —OR5, —OR8, —CH₂OR7, —SO_{y}R6 or —SR6; and wherein
R5 is alkyl of 1-4 carbons or aryl, preferably phenyl or naphthyl;
R6 is H, alkyl of 1-4 carbons, aryl of 6-10 carbons, preferably phenyl or naphthyl, or heteroaryl;
R7 is H or alkyl of 1-4 carbons;
R8 is the residue of an amino acid after the hydroxyl group of the carboxyl group is removed;
n is an integer of 1-4; and
y is 1 or 2, with the proviso that when R1 is —CH₂)₃OH and R2 is —H, then R3 cannot be —CH₂OH,
—CH₂OCH₂CH₃, or —CH₂SCH₂CH₃.

4. The pharmaceutical delivery system of claim 1, wherein the fused pyrrolocarbazole is one or more compounds defined by Formula II and salts thereof and prodrugs thereof and mixtures of the compounds, salts and prodrugs thereof: 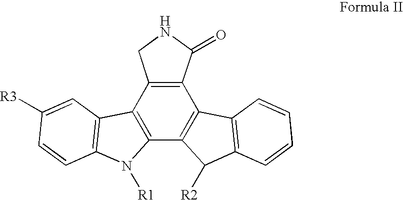
R₁ and R₂ are the same or different and are independently selected from —H, or alkyl of 1-8 carbons, substituted with —H, —OH or —OR4 where R4 is an alkyl of 1-4 carbons, aryl or the residue of an amino acid after the hydroxyl group of the carboxyl group is removed; and R3 is —CH₂OH; —CH₂OR7; —(CH₂)ₙSR5; —(CH₂)ₙSO_{y}R5; —CH₂SR₅; or alkyl of 1-8 carbons substituted with —OH, —OR5, —OR8, —CH₂OR7, —S(O)_{y}R6 or —SR6; and wherein R5 is alkyl of 1-4 carbons or aryl; R6 is H, alkyl of 1-4 carbons or aryl of 6-10 carbons; R7 is H or alkyl of 1-4 carbons; R8 is the residue of an amino acid after the hydroxyl group of the carboxyl group is removed; n is an integer of 1-4; and y is 1 or 2.

5. The pharmaceutical delivery system of claim 4, wherein R1 is an alkyl of 1-4 carbons, substituted with —OH or —OR4 wherein R4 is the residue of an amino acid after the hydroxyl group of the carboxyl group is removed; R2 is —H; and R3 is alkyl of 1-4 carbons, substituted with —OR5, —OR8, —CH₂OR7, —S(O)_{y}R6 or —SR8; and wherein R5 is alkyl of 1-4 carbons or aryl; R6 is H, alkyl of 1-4 carbons or aryl of 6-10 carbons; R7 is H or alkyl of 1-4 carbons; and R8 is the residue of an amino acid after the hydroxyl group of the carboxyl group is removed.

6. The pharmaceutical delivery system of claim 4, wherein R1 is —CH₂CH₂CH₂OH or —CH₂CH₂CH₂OCOCH₂N(CH₃)₂; R2 is H; and R3 is —CH₂OR7 wherein R7 is alkyl of 1-4 carbons.

7. The pharmaceutical delivery system of claim 1 wherein the fused pyrrolocarbazole is selected from the groups consisting of compounds represented in Table I and salts thereof and prodrugs thereof and mixtures of the salts and prodrugs thereof:
TABLE 1

| CMPD | | | |
|---|---|---|---|
| NO | R1 | R2 | R3 |
| 1 | —CH₂CH₂CH₂OH | —H | —CH₂OCH₃ |
| 2 | —CH₂CH₂CH₂OH | —H | —CH₂OCH(CH₃)₂ |
| 3 | —CH₂CH₂CH₂OH | —H | —CH₂O— |
| | | | CH(CH₃)CH₂CH₃ |
| 4 | —CH₂CH₂CH₂OH | —H | (S) —CH₂O— |
| | | | CH(CH₃)CH₂CH₃ |
| 5 | —CH₂CH₂CH₂OH | —H | (R) —CH₂O— |
| | | | CH(CH₃)CH₂CH₃ |
| 6 | —CH₂CHOHCH₃ | —H | —CH₂OCH₂CH₃ |
| 7 | —CH₂CH₂CH₂OH | —H | —CH₂OCH₂CH₂CH₃ |
| 8 | —CH₂CH₂CH₂OH | —H | —CH₂OCH₂CH₂ |
| | | | CH₂CH₃ |
| 9 | —CH₂CH₂CH₂OH | —H | —CH(CH₃)OCH₂CH₃ |
| 10 | —CH₂CH₂CH₂OH | —H | (chiral) |
| | | | —CH(CH₃)OCH₂CH₃ |
| 11 | —CH₂CH₂CH₂OH | —H | (chiral) |
| | | | —CH(CH₃)OCH₂CH₃ |
| 12 | —CH₂CH₂CH₂OH | —H | —CH(CH₃)OCH₃ |
| 13 | —H | | —CH₂OCH₂CH₃ |
| 14 | —CH₂CH₂CH₂OH | —H | —CH(CH₃)O— |
| | | | CH₂CH₂CH₂CH₃ |
| 15 | —CH₂CH₂CH₂OH | —H | —CH(CH₃)O— |
| | | | CH(CH₃)₂ |
| 16 | —CH₂CH₂CH₂OH | —H | —CH₂OC(CH₃)₃ |
| 17 | —CH₂CH₂CH₂OCO— | —H | —CH₂OCH(CH₃)₂ |
| | CH₂NH₂ | | |
| 18 | —CH₂CH₂CH₂OCO— | —H | —CH₂OCH(CH₃)₂ |
| | CH₂NH₂— | | |
| | CH₂CH₂CH₂CH₂NH₂ | | |
| 19 | —CH₂CH₂CH₂OCOCH₂ | —H | —CH₂OCH(CH₃)₂ |
| | —CH₂NH₂ | | |
| 20 | —CH₂CH₂CH₂OCOCH₂ | —H | —CH₂OCH(CH₃)₂ |
| | —CH₂CH₂N(CH₃)₂ | | |
| 21 | —CH₂CH₂CH₂OCO— | —H | —CH₂OCH(CH₃)₂ |
| | CH₂N(CH₂)₂ | | |
| 22 | —CH₂CH₂CH₂OCO— | —H | —CH₂OCH(CH₃)₂ |
| | CH₂CH₂CH₂ | | |
| 23 | —CH₂CH₂OH | —H | —CH₂SCH₂CH₃ |
| 24 | —CH₂CH₂CH₂OH | —H | —CH₂SCH₂CH₃ |
| 25 | —CH₂CH₂CH₂OH | —H | —CH₂S(O)CH(CH₃)₂ |
| 26 | —CH₂CH₂OH | —H | —CH₂OH |
| 27 | —H | —H | —CH₂OH |
| 28 | —H | —H | —CH₂OCH₂CH₃ |
| 29 | —H | —H | —CH₂OCH(CH₃)₂ |
| 30 | —CH₂CH₂CH₂OH | —H | —CH(OH)CH₃ |
| 31 | —CH₂CH₂CH₂OH | —H | —CH(OH)CH₂CH₃ |
| 32 | —H | —H | —CH(OH)CH₃ |
| 33 | —H | —H | (+/−) —CH(OCH₃)CH₃ |
| 34 | —CH₂CH₂CH₂OH | —CH₂OH | —CH₂OCH(CH₃)₂ |

8. The pharmaceutical delivery system of claim 1, wherein the fused pyrrolocarbazole is of the following formula and salts thereof and prodrugs thereof and mixtures of the compound, salts and prodrugs therof:

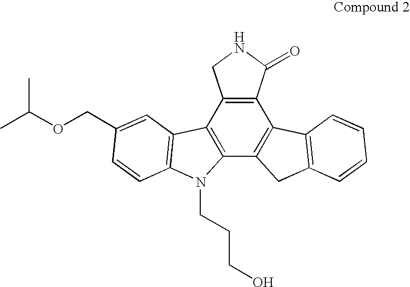

9. The pharmaceutical delivery system of claim 1, wherein the fused pyrrolocarbazole is a compound of the following formula and salts thereof and prodrugs thereof and mixtures of the compound, salts and/or prodrugs thereof:

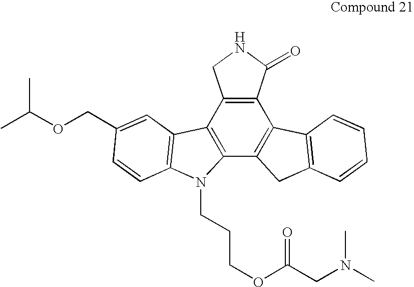

10. The pharmaceutical delivery system of claim 1, wherein the delivery device is a drug reservoir delivery device.

11. The pharmaceutical delivery system of claim 10, wherein the drug reservoir delivery device has a drug impermeable portion that surrounds at least a portion of a drug core comprising a fused pyrrolocarbazole, wherein the drug impermeable portion defines a reservoir and restricts flow of fused pyrrolocarbazole from the reservoir.

12. The pharmaceutical delivery system of claim 10, wherein the drug impermeable portion defines a wall, layer or coating.

13. The pharmaceutical delivery system of claim 11, wherein the drug impermeable portion is made of silicone.

14. The pharmaceutical delivery system of claim 11, further comprising a drug permeable portion that surrounds at least a portion of a drug core, wherein the drug impermeable portion further defines the reservoir and allows passage of fused pyrrolocarbazole through the drug permeable portion.

15. The pharmaceutical delivery system of claim 14, wherein the drug permeable portion defines a wall, layer or coating.

16. The pharmaceutical delivery system of claim 14, wherein the drug permeable portion is made of silicone.

17. The pharmaceutical delivery system of claim 1, wherein the pharmaceutical delivery device comprises:
a holder made of a material impermeable to passage of a pharmaceutical agent and including at least one opening for passage of the active agent therethrough;
a drug core contained into which the fused pyrrolocarbazole is received; and
a preformed disc made of an expandable material permeable to passage of the fused pyrrolocarbazole, the disc contained in the holder and disposed between the drug core and the at least one opening in the holder,
wherein a groove is formed in the holder in the vicinity of the disc.

18. The pharmaceutical delivery system of claim 1, wherein the system is configured to maintain the concentration of fused pyrrolocarbazole in the vitreous that is a minimum of about 10 ng/ml.

19. The pharmaceutical delivery system of claim 1, that is configured to maintain the effective concentration of fused pyrrolocarbazole in the vitreous that is at least about 50 times greater than the concentration of the fused pyrrolocarbazole in the blood of the patient.

20. The pharmaceutical delivery system of claim 1, wherein the effective concentration of fused pyrrolocarbazole in the vitreous of is maintained for a minimum of 6 weeks.

21. The pharmaceutical delivery system of claim 1, wherein the anti-angiogenesis agent is released from the pharmaceutical delivery system at rate that is a minimum of about 5 ng per day and a maximum of about 1 mg per day.

22. A method for treating angiogenic disorders in the eye of a patient, which comprises administering to a host in need of such treatment a pharmaceutical delivery system comprising a pharmaceutical delivery device selected from the group consisting of a reservoir device and a drug infusion device, wherein the pharmaceutical delivery device is sized and configured to be inserted into the eye of a patient and a therapeutically effective amount of a fused pyrrolocarbazole.

23. The method of claim 22, wherein the fused pyrrolocarbazole is selected from the group consisting of an indolocarbazole and an indenocarbazole and mixtures thereof.

24. The method of claim 22, wherein the fused pyrrolocarbazole is defined by the following Formula I and salts thereof and prodrugs thereof: 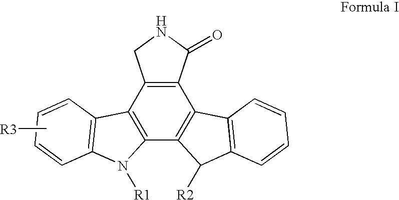
R1 and R2 are the same or different and are independently selected from H, or alkyl of 1-8 carbons, substituted with —OH, or —OR4 where R4 is an alkyl of 1-4 carbons, aryl or the residue of an amino acid after the hydroxyl group of the carboxyl group is removed; and R3 is —CH₂OH; —CH₂OR7; —(CH₂)ₙSR5; —(CH₂)ₙS(O)_{y}R5; —CH₂SR5; or alkyl of 1-8 carbons substituted with —OH, —OR5, —OR8, —CH₂OR7, —S(O)_{y}R6 or —SR8; and wherein R5 is alkyl of 1-4 carbons or aryl; R6 is H, alkyl of 1-4 carbons or aryl of 6-10 carbons; R7 is H or alkyl of 1-4 carbons; R8 is the residue of an amino acid after the hydroxyl group of the carboxyl group is removed; n is an integer of 1-4; and y is 1 or 2; with the proviso that when R1 is (CH₂)₃OH and R2 is H, then R3 cannot be —CH₂OH, alkyl of 1-8 carbons substituted with —OH or —SR8 , wherein R6 is alkyl of 1-4 carbons; —(CH₂)ₙSR5, wherein n is 1 and R5 is alkyl of 1-4 carbons; or —CH₂SR5, wherein R5 is alkyl of 1-4 carbons.

25. The method of claim 22, wherein the fused pyrrolocarbazole is defined by the following Formula II and salts thereof and prodrugs thereof: 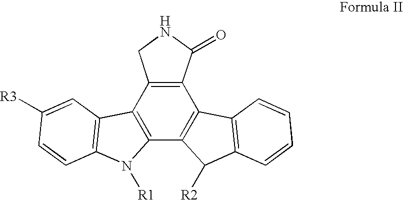
R₁ and R₂ are the same or different and are independently selected from H, or alkyl of 1-8 carbons, substituted with —H, —OH or —OR4 where R4 is an alkyl of 1-4 carbons, aryl or the residue of an amino acid after the hydroxyl group of the carboxyl group is removed; and R3 is —CH₂OH; —CH₂OR7; —(CH₂)ₙSR5; —(CH₂)ₙSO_{y}R5; —CH₂SR₅; or alkyl of carbons substituted with —OH, —OR5, —OR8, —CH₂OR7, —S(O) yR6 or —SR6; and wherein R5 is alkyl of 1-4 carbons or aryl; R6 is H, alkyl of 1-4 carbons or aryl of 6-10 carbons; R7 is H or alkyl of 1-4 carbons; R8 is the residue of an amino acid after the hydroxyl group of the carboxyl group is removed; n is an integer of 1-4; and y is 1 or 2; with the proviso that when R1 is —(CH₂)₃OH and R2 is —H, then R3 cannot be —CH₂OH, —CH₂OCH₂CH₃, or —CH₂SCH₂CH₃.

26. The method of claim 25, wherein R1 is an alkyl of 1-4 carbons, substituted with —OH or —OR4 wherein R4 is the residue of an amino acid after the hydroxyl group of the carboxyl group is removed; R2 is H; and R3 is alkyl of 1-4 carbons, substituted with —OR5, —OR8, —CH₂OR7, —S(O)_{y}R6 or —SR8; and wherein R5 is alkyl of 1-4 carbons or aryl; R6 is H, alkyl of 1-4 carbons or aryl of 6-10 carbons; R7 is H or alkyl of 1-4 carbons; and R8 is the residue of an amino acid after the hydroxyl group of the carboxyl group is removed.

27. The method of claim 25, wherein R1 is —CH₂CH₂CH₂OH or —CH₂CH₂CH₂OCOCH₂N(CH₃)₂; R2 is H; and R3 is —CH₂OR7 wherein R7 is alkyl of 1-4 carbons.

28. The method of claim 22, wherein the fused pyrrolocarbazole is selected from the group consisting of the compounds represented in Table I and salts thereof and prodrugs thereof and mixtures of such compounds, salts and/or prodrugs thereof:

| CMPD | | | |
|---|---|---|---|
| NO | R1 | R2 | R3 |
| 1 | —CH₂CH₂CH₂OH | —H | —CH₂OCH₃ |
| 2 | —CH₂CH₂CH₂OH | —H | —CH₂OCH(CH₃)₂ |
| 3 | —CH₂CH₂CH₂OH | —H | —CH₂O— |
| | | | CH(CH₃)CH₂CH₃ |
| 4 | —CH₂CH₂CH₂OH | —H | (S) —CH₂O— |
| | | | CH(CH₃)CH₂CH₃ |
| 5 | —CH₂CH₂CH₂OH | —H | (R) —CH₂O— |
| | | | CH(CH₃)CH₂CH₃ |
| 6 | —CH₂CHOHCH₃ | —H | —CH₂OCH₂CH₃ |
| 7 | —CH₂CH₂CH₂OH | —H | —CH₂OCH₂CH₂CH₃ |
| 8 | —CH₂CH₂CH₂OH | —H | —CH₂OCH₂CH₂ |
| | | | CH₂CH₃ |
| 9 | —CH₂GH₂CH₂OH | —H | —CH(CH₃)OGH₂CH₃ |
| 10 | —CH₂CH₂CH₂OH | —H | (chiral) |
| | | | —CH(CH₃)OCH₂CH₃ |
| 11 | —CH₂CH₂CH₂OH | —H | (chiral) |
| | | | —CH(CH₃)OCH₂CH₃ |
| 12 | —CH₂CH₂CH₂OH | —H | —CH(CH₃)OCH₃ |
| 13 | —H | | —CH₂OCH₂CH₃ |
| 14 | —CH₂CH₂CH₂OH | —H | —CH(CH₃)O— |
| | | | CH₂CH₂CH₂CH₃ |
| 15 | —CH₂CH₂CH₂OH | —H | —CH(CH₃)O— |
| | | | CH(CH₃)₂ |
| 16 | —CH₂CH₂CH₂OH | —H | —CH₂OC(CH₃)₃ |
| 17 | —CH₂CH₂CH₂OCO— | —H | —CH₂OCH(CH₃)₂ |
| | CH₂NH₂ | | |
| 18 | —CH₂CH₂CH₂OCO— | —H | —CH₂OCH(CH₃)₂ |
| | CH₂NH₂— | | |
| | CH₂CH₂CH₂CH₂NH₂ | | |
| 19 | —CH₂CH₂CH₂OCOCH₂ | —H | —CH₂OCH(CH₃)₂ |
| | —CH₂NH₂ | | |
| 20 | —CH₂CH₂CH₂OCOCH₂ | —H | —CH₂OCH(CH₃)₂ |
| | —CH₂CH₂N(CH₃)₂ | | |
| 21 | —CH₂CH₂CH₂OCO— | —H | —CH₂OCH(CH₃)₂ |
| | CH₂N(CH₂)₂ | | |
| 22 | —CH₂CH₂CH₂OCO— | —H | —CH₂OCH(CH₃)₂ |
| | CH₂CH₂CH₃ | | |
| 23 | —CH₂CH₂OH | —H | —CH₂SCH₂CH₃ |
| 24 | —CH₂CH₂CH₂OH | —H | —CH₂SCH₂CH₃ |
| 25 | —CH₂CH₂CH₂OH | —H | —CH₂S(O)CH(CH₃)₂ |
| 26 | —CH₂CH₂OH | —H | —CH₂OH |
| 27 | —H | —H | —CH₂OH |
| 28 | —H | —H | —CH₂OCH₂CH₃ |
| 29 | —H | —H | —CH₂OCH(CH₃)₂ |
| 30 | —CH₂CH₂CH₂OH | —H | —CH(OH)CH₃ |
| 31 | —CH₂CH₂CH₂OH | —H | —CH(OH)CH₂CH₃ |
| 32 | —H | —H | —CH(OH)CH₃ |
| 33 | —H | —H | (+/−) —CH(OCH₃)CH₃ |
| 34 | —CH₂CH₂CH₂OH | —CH₂OH | —CH₂OCH(CH₃)₂ |

29. The method of claim 22, wherein the fused pyrrolocarbazole is a compound of the following formula and salts thereof and prodrugs thereof and mixtures of the compound, salts and/or prodrugs:

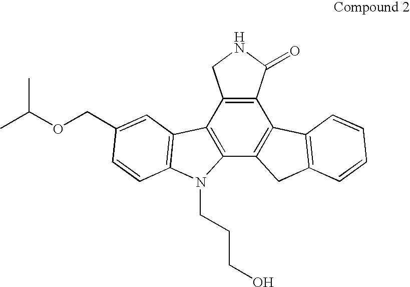

30. The method of claim 22, wherein the fused pyrrolocarbazole is of the following formula and salts and prodrugs thereof and mixtures of the compound, salts and/or prodrugs thereof:

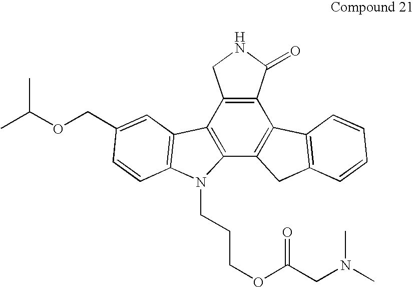

31. The method of claim 22, wherein the delivery device is a drug reservoir delivery device.

32. The method of claim 31, wherein the drug reservoir delivery device has a drug impermeable portion that surrounds at least a portion of a drug core comprising a fused pyrrolocarbazole, wherein the drug impermeable portion defines a reservoir and restricts flow of fused pyrrolocarbazole from the reservoir.

33. The method of claim 32, wherein the drug impermeable portion defines a wall, layer or coating.

34. The method of claim 32, wherein the drug impermeable portion is made of silicone.

35. The method of claim 32, further comprising a drug permeable portion that surrounds at least a portion of a drug core, wherein the drug permeable portion further defines the reservoir and allows passage of fused pyrrolocarbazole through the drug permeable portion.

36. The method of claim 32, wherein the drug permeable portion defines a wall, layer or coating.

37. The method of claim 32, wherein the drug permeable portion is made of poly(vinyl alcohol).

38. The method of claim 32 wherein the pharmaceutical delivery system comprises:
a holder made of a material impermeable to passage of a pharmaceutical agent and including at least one opening for passage of the active agent therethrough;
a drug core contained in the holder and including fused pyrrolocarbazole; and
a preformed disc made of an expandable material permeable to passage of the fused pyrrolocarbazole, the disc contained in the holder and disposed between the drug core and the at least one opening in the holder,
wherein a groove is formed in the holder in the vicinity of the disc.

39. The method of claim 22, wherein the pharmaceutical delivery system is configured to maintain the concentration of fused pyrrolocarbazole in the vitreous that is a minimum of about 10 ng/ml.

40. The method of claim 22, that is configured to maintain the effective concentration of fused pyrrolocarbazole in the vitreous that is at least about 50 times greater than the concentration of the fused pyrrolocarbazole in the blood of the patient.

41. The method of claim 22, wherein the effective concentration of fused pyrrolocarbazole in the vitreous of is maintained for a minimum of 6 weeks.

42. The method of claim 22, wherein the anti-angiogenesis agent is released from the pharmaceutical delivery system at rate that is a minimum of about 5 ng per day and a maximum of about 1 mg per day.

43. The method of claim 22, wherein the anti-angiogenesis agent is released from the pharmaceutical delivery system at rate that is a minimum of about 5 ng per day and a maximum of about 1 mg per day.

44. A method for treating an inflammatory disorder in the eye of a patient, which comprises administering to a host in need of such treatment a pharmaceutical delivery system comprising pharmaceutical delivery device selected from the group consisting of reservoir devices and drug infusion device and a therapeutically effective amount of a fused pyrrolocarbazole.

45. The method of claim 44, wherein the pharmaceutical delivery system is configured to maintain the concentration of fused pyrrolocarbazole in the vitreous that is a minimum of about 10 ng/ml.

46. The method of claim 44, that is configured to maintain the effective concentration of fused pyrrolocarbazole in the vitreous that is at least about 50 times greater than the concentration of the fused pyrrolocarbazole in the blood of the patient.

47. The method of claim 44, wherein the effective concentration of fused pyrrolocarbazole in the vitreous of is maintained for a minimum of 6 weeks.

48. The method of claim 44, wherein the inflammatory disorder is edema.
